(19) United States
(12) Patent Application Publication
(10) Pub. No.: US 2003/0157559 A1
(43) Pub. Date: Aug. 21, 2003

(54) Analyzing method for non-uniform-density sample and device and system thereof

(76) Inventors: Kazuhiko Omote, Tokyo (JP); Alexander Ulyanenkov, Tokyo (JP); Shigeru Kawamura, Yamanashi (JP)

Correspondence Address: WENDEROTH, LIND & PONACK, L.L.P., 2033 K STREET N. W., SUITE 800, WASHINGTON, DC, 20006-1021, US

(21) Appl. No.: 10/240,671

(22) PCT Filed: Mar. 30, 2001
(86) PCT No.: PCT/JP01/02819

(30) Foreign Application Priority Data
Apr. 4, 2000 (JP) 2000-102781
Mar. 26, 2001 (JP) 2001-088656

(51) Int. Cl. G01N 33/53

(52) U.S. Cl. 435/7.1

(57) ABSTRACT

A novel non-uniform-density sample analyzing method capable of analyzing simply and highly accurately the distribution state of particle-like matter in a non-uniform-density sample such as a thin film and bulk element and a non-uniform-density sample analyzing device and a non-uniform-density sample analyzing system for implementing the method are provided; said method comprising the steps of calculating a simulated X-ray scattering curve under the same conditions as measuring conditions for an actually measured X-ray scattering curve by using a scattering function that simulates an X-ray scattering curve according to a fitting parameter indicating distribution state of particle-like matter, carrying out fitting between the simulated X-ray scattering curve and the actually measured X-ray scattering curve while changing the fitting parameter, and using, as the distribution state of particulate matters in the non-uniform-density sample, the value of the fitting parameter when the simulated X-ray scattering curve agrees with the actually measured X-ray scattering curve.

### TECHNICAL FIELD

The invention of this application relates to an analyzing method for a non-uniform-density sample and a device and system thereof. More specifically, the invention of this application to a non-uniform-density sample analyzing method, a non-uniform-density sample analyzing device and a non-uniform-density sample analyzing system which are capable of analyzing simply and highly accurately the distribution state of particle-like matter in a non-uniform-density sample and are useful for evaluation of the density non-uniformity of such a thin film, a bulk body and the like.

### BACKGROUND ART

In a thin film or a bulk body produced for various purposes, often, there are undesired particle-like matter mixed unintentionally or particle-like matter mixed intentionally. With distribution of this particle-like matter, the thin film and the bulk body come to have non-uniform density. Further, in the thin film, its particle diameter may sometimes become uneven depending on film forming methods. It is very important, irrespective of the type of various utilization fields, to evaluate the density non-uniformity for formation and usage of such non-uniform-density thin film or non-uniform-density bulk body. For example, generally in case of intentional particle-like matter, it is considered desirable that each particle diameter is the same as much as possible and the evaluation of the density non-uniformity is indispensable to achieve this.

In order to evaluate the density non-uniformity, it is necessary to objectively analyze the distribution state of particle-like matter, such as a size of particle-like matter and a size of its distribution region (that is, region non-uniform in density). For example, conventionally, as the method for analyzing the density non-uniformity or the diameter of a pore, there have been known methods such as a gas absorption method which analyzes the size of the particle-like matter and the size of the distribution region based on time of absorbing nitrogen gas and a X-ray small-angle scattering method which analyzes the size of the distribution region by using a phenomenon in which X-ray in scattered within a range from 0° to several degrees of the scattering angle.

However, there are problems that the gas absorption method takes long time for its measurement and further is not capable of performing the measurement for the pore which gas cannot permeate. And as for the conventional X-ray small-angle scattering method, there are problems such that a thin film on a substrate needs to be separated from the substrate before its measurement because the measurement is usually executed by passing through a sample and thus the density non-uniformity of the thin film on the substrate cannot be analyzed accurately.

Therefore, there have been great demands for realization of an analyzing method for non-uniform-density sample which is capable of analyzing distribution state of particle-like matter without any destruction and in a short time and is applicable to various types of non-uniform-density thin film or non-uniform-density bulk body. Further, reductionizing of particle-like matter has been accelerated as a more advanced function has been pursued, so that the necessity of analyzing size of the particle-like matter of less than several nanometers and the size of its distribution region has been increased.

The invention of this application has been invented in views of the foregoing circumstances, and an object of the invention of this application is to provide a novel non-uniform-density sample analyzing method, a novel non-uniform-density sample analyzing device and a novel nor-uniform-density sample analyzing system which are capable of solving the problems of the conventional technology and analyzing distribution state of particle-like matter in a non-uniform-density sample easily at a high accuracy.

### DISCLOSURE OF THE INVENTION

In order to solve the forgoing problems, the invention of this application provides a non-uniform-density sample analyzing method for analyzing distribution state of particle-like matter in a non-uniform-density sample, comprising: computing a simulated X-ray scattering curve or a simulated particle bean scattering curve under the same condition as a measuring condition of an actually measured X-ray scattering curve or an actually measured particle beam scattering curve by using a scattering function expressing a X-ray scattering curve or the particle beam scattering curve according to a fitting parameter indicating distribution state of particle-like matter; and carrying out fitting between the simulated X-ray scattering curve and the actually measured X-ray scattering curve or fitting between the simulated particle beam scattering curve and the actually measured particle beam scattering curve while changing the fitting parameter, wherein the value of the fitting parameter when the simulated X-ray scattering curve agrees with the actually measured X-ray scattering curve or the value of the fitting parameter when the simulated particle beam scattering curve agrees with the actually measured particle beam scattering serves to indicate the distribution state of the particle-like matter in the non-uniform-density sample (claim 1) (claim 2). The invention of this application also provides the non-uniform-density sample analyzing method: wherein the fitting parameter indicates an average particle diameter and distribution shape of particle-like matter and the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the average particle diameter and distribution shape of particle-like matter in the non-uniform-density sample (claim 3); wherein the fitting parameter indicates a nearest distance and correlation coefficient between the particle-like matter and the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the nearest distance and correlation coefficient between the particle-like matter in the non-uniform-density sample (claim 4); wherein the fitting parameter indicates a content ratio and correlation distance of the particle-like matter and the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the content ratio and correlation distance of the particle-like matter in the non-uniform-density sample (claim 5); wherein the actually measured X-ray scattering curve or the actually measured particle beam scattering curve is measured under any condition selected from the condition of θin=θout±offset angle Δω, condition of scanning θout with θin constant and condition for scanning θin with θout constant and the simulated X-ray scattering curve or the simulated particle beam scattering curve is computed according to the scattering function under the same condition as that measuring condition (claim 6); and wherein a function which employs absorption/irradiating area correction taking into account at least one of refraction, scattering and reflection or particle-like matter correlation function or both of them is used as the scattering function.

Further, the invention of this application provides a non-uniform-density sample analyzing device for analyzing distribution state of particle-like matter in a non-uniform-density sample, comprising: a function storage means for storing a scattering function expressing a X-ray scattering curve or a particle beam scattering curve according to a fitting parameter indicating distribution state of particle-like matter; a simulating means for computing a simulated X-ray scattering curve or a simulated particle beam scattering curve under the same condition as a measuring condition of an actually measured X-ray scattering curve or an actually measured particle beam scattering curve by using the scattering function from the function storage means; and a fitting means for carrying out fitting between the simulated X-ray scattering curve and the actually measured X-ray scattering curve or fitting between the simulated X-ray scattering curve and the actually measured particle beam scattering curve while changing the fitting parameter, wherein the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the distribution state of the particle-like matter in the non-uniform-density sample (claim 10) (claim 11). The invention of this application also provides the non-uniform-density sample analyzing device: wherein when the actually measured X-ray scattering curve or the actually measured particle beam scattering curve is measured under any condition selected from the condition of θin=θout±offset angle Δω, condition of scanning θout with θin constant and condition of scanning θin with θout constant, the simulating means computes the simulated X-ray scattering curve or the simulated particle beam scattering curve with the scattering function under the same condition as that measuring condition (claim 12); wherein the function storage means stores, as the scattering function, a function which employs absorption/irradiating area correction taking into account at least one of refraction, scattering and reflection or particle-like matter correlation function or both of them (claim 13).

Furthermore, the invention of this application provides a non-uniform-density sample analyzing system for analyzing distribution state of particle-like matter in a non-uniform-density sample, comprising a X-ray measuring device for measuring an actually measured X-ray scattering curve in the non-uniform-density sample or a particle beam measuring device for measuring an actually measured particle beam scattering curve in the non-uniform-density sample, and the aforementioned non-uniform-density sample analyzing device, wherein the actually measured X-ray scattering curve by the X-ray measuring device or the actually measured particle beam scattering curve by the particle beam measuring device and various kinds of parameters at the measurement necessary for computing the scattering function are made available by the non-uniform-density sample analyzing device (claim 16) (claim 17).

Furthermore, the invention of this application provides a non-uniform-density sample analyzing method for analyzing distribution state of particle-like matter in a non-uniform-density sample, characterized in that if the non-uniform-density sample is porous film, the distribution state of the particle-like matter in the porous film is analyzed using a measuring result of the X-ray scattering curve (claim 18).

Moreover, the foregoing respective analyzing method, analyzing device and analyzing system can handle a thin film or a bulk body which is a non-uniform-density sample, as an analyzing object (claim 8) (claim 14). A porous film can be an example of the thin film. In case of the porous film, the particle-like matter is fine particle or pore which forms the porous film (claim 9) (claim 15).

### BRIEF DESCRIPTION OF DRAWINGS

FIG. 1 is a flow chart showing an example of analyzing procedure according to the non-uniform-density sample analyzing method of the invention of this application;

FIGS. 2(a), (b) are diagrams exemplifying a spherical model and a cylindrical model in the non-uniform-density form factor, respectively;

FIG. 3 is a diagram exemplifying the states of refraction, reflection and scattering of X-ray in the non-uniform-density thin film;

FIG. 4 is a diagram showing an example of a slit function;

FIG. 5 is a major portion block diagram exemplifying the non-uniform-density sample analyzing device and system of the invention of this application. Respective reference numerals indicate non-uniform-density sample analyzing system (1), X-ray measuring device (2), non-uniform-density sample analyzing device (3), critical angle acquisition means (31), function storage means (32), simulation means (33), fitting means (34) and output means (35), (36);

FIG. 6 is a diagram showing an example of gamma distributions;

FIG. 7 is a diagram showing another example of gamma distributions;

FIG. 8 is a diagram exemplifying simulated X-ray scattering curves;

FIG. 9 in a diagram exemplifying simulated X-ray scattering curves;

FIG. 10 is a diagram exemplifying measuring results of X-ray reflectivity curve and X-ray scattering curve as one example;

FIG. 11 is a diagram showing simulated X-ray scattering curves and actually measured X-ray scattering curves overlaying on each other as one example;

FIG. 12 is a diagram exemplifying distribution of the pore size of porous film as one example;

FIG. 13 is a diagram showing simulated X-ray scattering curves and an actually measured X-ray scattering curve overlaying on each other as another example; and

FIG. 14 is a diagram showing a simulated X-ray scattering curve and an actually measured X-ray scattering curve overlaying on each other as still another example.

### BEST MODE FOR CARRYING OUT THE INVENTION

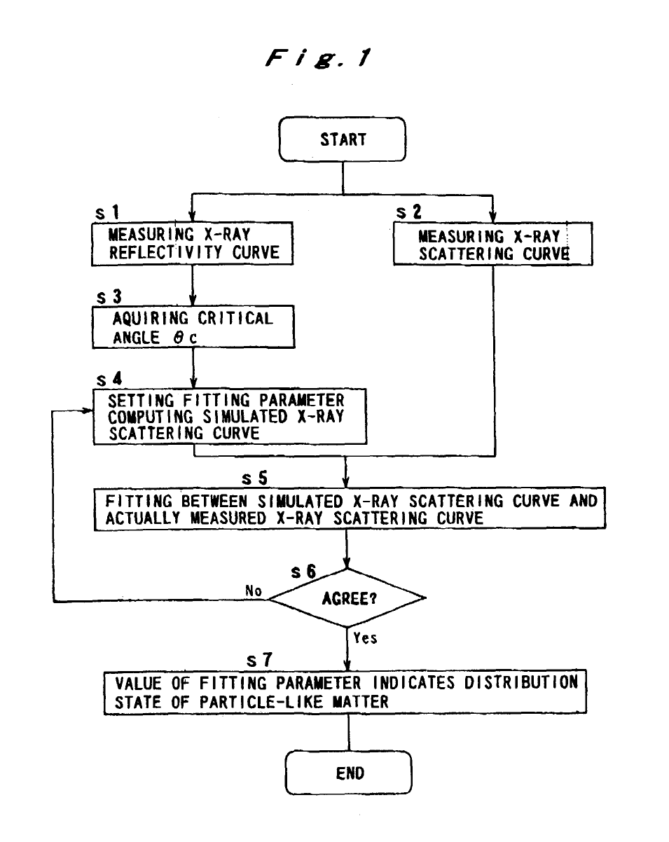

Hereinafter, the embodiment of the invention of this application will be described with reference to FIG. 1. FIG. 1 is a flow chart showing an example of analyzing procedure based on the non-uniform-density sample analyzing method of the invention of this application. The analyzing method using X-ray will be described mainly.

<Steps s1, s2> According to the invention of this application, a simulated X-ray scattering curve is computed using a scattering function expressing a X-ray scattering curve according to a fitting parameter indicating distribution state of particle-like matter. As described later, this scattering function may employ a fitting parameter [Ro, M] indicating average particle diameter and distribution shape in case where the particle-like matter is modeled by a spherical model, a fitting parameter [D, a] indicating diameter and aspect ratio in case where the particle-like matter in modeled by a cylindrical model, a fitting parameter [L, η] indicating nearest distance and correlation coefficient of the particle-like matter or a fitting parameter [P, ζ] indicating content ratio and correlation distance of the particle-like grain.

Any scattering function needs X-ray reflectivity curve, X-ray scattering curve and respective values introduced from these curves. Thus, prior to simulation and fitting, the X-ray reflectivity curve and X-ray scattering curve of such non-uniform-density substance as thin film, bulk body in which the particle-like matter is distributed are measured.

<Step s1)> The X-ray reflectivity curve is measured in the condition of X-ray incident angle θin=X-ray emission angle θout (that is, mirror reflection). The X-ray incident angle θin indicates an X-ray incident angle on the surface of the non-uniform-density sample and the X-ray emission angle θout indicates an X-ray emission angle on the surface of the non-uniform-density sample.

<Step s2> The X-ray scattering curve is measured under the condition of X-ray incident angle θin=X-ray emission angle θout-offset Δω or under the condition of X-ray incident angle θin=X-ray emission angle θout+offset Δω or under both the conditions (hereinafter these conditions are called θin±θout±Δω). The offset Δω mentioned here refers to a difference in angle between θin and θout. In case of Δω =0°, it comes that θin=θout thereby producing mirror reflection, so that the same thing as measurement of X-ray reflectivity occurs. The measurement of the X-ray scattering curve is carried out in the condition that this Δω in deflected slightly from 0° (offset). Δω is desired to be as near 0° as possible and further, a value which reduces influence of strong mirror reflection when Δω=0°.

Because the measurement of X-ray scattering curve under θin=θout±Δω is just measurement of diffuse scattering and this diffuse scattering originates from existence of the particle-like matter in the thin film or bulk body or originates from non-uniformity of density of the non-uniform-density sample, the non-uniformity of density of the non-uniform-density sample such an the thin film, bulk body can be analyzed accurately by fitting to the simulation scattering curve computed from the actually measured X-ray scattering curve and respective kinds of functions described above.

The X-ray scattering curve may be measured in the condition of scanning the X-ray scattering angle θout by making the X-ray incident angle θin constant or conversely in the condition of scanning the X-ray incident angle θin by making the X-ray emission angle θout constant. In this case also, measurement of the diffuse scattering necessary for high precision simulation and fitting can be carried out.

<Step s3> Because the respective scattering functions described later employ critical angle θc of the non-uniform-density sample, critical angle θc is obtained directly from a measured X-ray reflection curve first. The critical angle θc of the X-ray reflection curve can be determined according to a well-known method. Specifically, an angle in which reflectivity (reflecting X-ray intensity) drops rapidly in the X-ray reflectivity curve comes to the critical angle θc. In fact, there in relationship of θc−√(2δ) and n=1−δ in the critical angle θc, the numerical value δ and the refractive index n.

On the other hand, if an element which constitutes the non-uniform-density sample is evident, an average density ρ of the non-uniform-density sample can be determined from δ. More specifically, if composition ratio cj, mass number Mj and atom scattering factor of the composition element j are evident, the average density ρ of the non-uniform-density sample can be determined by the following equation.

$$δ = \frac{{r}_{e}}{2 π} {λ}^{2} {N}_{A} \cdot ρ \cdot \frac{{\sum }_{j} {c}_{j} Re ( {f}_{j} )}{{\sum }_{j} {c}_{j} {M}_{j}} \qquad Eq . 1$$

rₑ:Classical electron radius ≅2.818×10⁻¹³ cm

N_{A}:Avogadro number≅6.022×10²³ mol⁻¹

ρ:Average density of non-uniform-density sample

cⱼ:Composition ratio of element j in non-uniform-density sample

Mⱼ:Atomic weight of element j in non-uniform-density sample

fⱼ:Atomic scattering factor of element j in non-uniform-density sample

The respective values necessary for computation can be estimated upon production of the non-uniform-density sample. The average density ρ of this non-uniform-density sample is very effective information for evaluation and production of the non-uniform-density sample as well as the distribution state including the particle diameter and distribution shape of the particle-like matter in the obtained non-uniform-density sample as described later.

<Step s4> According to the invention of this application, after the preliminary preparation for simulation and fitting is finished as described above, an arbitrary value of the fitting parameter is selected and a simulated X-ray scattering curve is computed under the same condition as measuring condition of the scattering curve (scanning of θout with θin=θout±Δω and θin constant or scanning of θin with θout constant) by using a scattering function indicating an X-ray scattering curve according to a fitting parameter indicating the distribution state of the particle-like matter.

More specifically, the following Eq.2 indicates an example of the scattering function and expresses all X-ray scattering curves at θin and θout excluding the mirror reflection of θin=θout.

$$\begin{matrix}I ( {θ}_{in} , {θ}_{out} ) = \int {\vert {F}_{s} ( q ; \{ p \} ) \vert}^{2} P ( \{ p \} ) ⅆ \{ p \} \\ \begin{matrix} & q = \frac{4 π sin ( \frac{\sqrt{{θ}_{i n}^{2} - 2 δ} + \sqrt{{θ}_{out}^{2} - 2 δ}}{2} )}{λ}\end{matrix} \\ \begin{matrix}I ( {θ}_{i n} , {θ}_{out} ) & : Scattering function \\ {F}_{s} ( q ; \{ p \} ) & : Non \text{-} uniform \text{-} density scattering form factor \\ q = \vert q \vert & : Magnitude of scattering vector \\ q & : Scattering vector \\ {θ}_{c} = \sqrt{2 δ} & : Critical angle \\ n = 1 - δ & : Index of refraction \\ λ & : X \text{-} ray wavelength \\ P ( \{ p \} ) & : Non \text{-} uniform \text{-} density distribution function \\ \{ p \} & : Group of distribution function parameters\end{matrix}\end{matrix} \qquad Eq . 2$$

In the scattering function given in the form of Eq. 2, the non-uniform-density scattering form factor is an important element for expressing the X-ray scattering curve. The non-uniform-density scattering form factor expresses the shape of the particle-like matter in the non-uniform-density sample with a specific shape model, thereby indicating that that shape model is distributed in a certain state in the sample, and according to this factor, the X-ray scattering curve which expresses an influence by the distribution of the particle-like matter can be simulated at a high freedom and high accuracy. Meanwhile, {p} which determines the non-uniform-density distribution function indicates that some groups of the parameters for determining the distribution functions may exist.

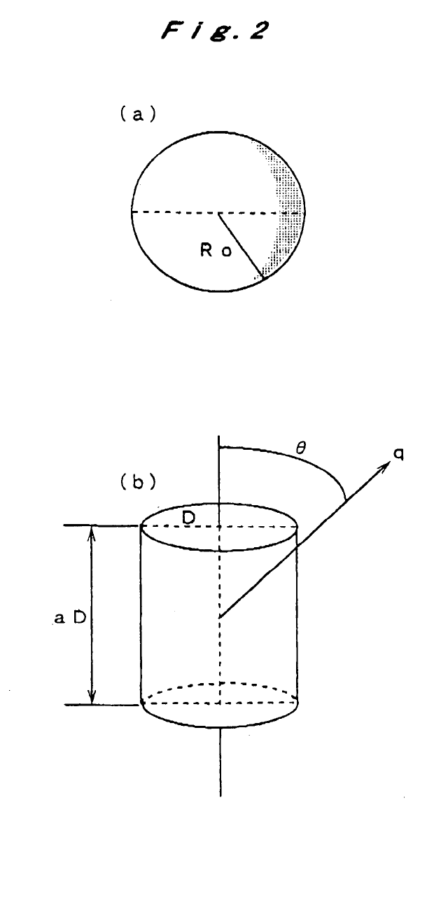

As the shape model of the particle-like matter, for example, the spherical model exemplified in FIG. 2(a) and the cylindrical model exemplified in FIG. 2(b) can be considered. The shape of every particle-like matter can be modeled by selecting one depending on an analyzing object.

First, the scattering function I(q) using the spherical model is given in the form of the following Eq.3 while the particle diameter distribution function indicating the particle diameter is given in the form of Eq.4and the particle form factor indicating the particle shape is given in the form of Eq.5. Incidentally, Eq.3 can be developed to the following Eq.6 by using Eq.4 and Eq.5. In this case, the parameter [Ro, M] indicating the average particle radius and distribution shape of the particle-like matter modeled based on the spherical model is a fitting parameter indicating the distribution state of the particle-like matter. The scattering function I(q) of the Eq.3 or Eq.6 can express various distribution states by selecting an arbitrary value [Ro, M] according to these fitting parameters and is a function expressing various kinds of the X-ray scattering curves affected by that distribution state.

$$I ( q ) = {\int }_{0}^{∞} ⅆ R \cdot {\vert {Ω}^{FT} ( q , R ) \vert}^{2} \cdot {P}_{{R}_{o}}^{M} ( R ) \cdot \frac{1}{{R}^{3}} {R}_{o}^{3} {ρ}_{o} \qquad Eq . 3$$

P_{R}_{ₒ}^{M}(R) :Particle radius distribution function

Rₒ:Average particle radius parameter

M:Distribution shape parameter

R:Integration variable

q=|q|:Magnitude of scattering vector

q:Scattering vector

ρₒ:Average density of particle-like matter

Ω^{FT}(q,R):Particle form factor

$$\begin{matrix}{P}_{Ro}^{M} ( R ) = \frac{{( \frac{M}{{R}_{o}} )}^{M}}{Γ ( M )} \cdot {ⅇ}^{- \frac{MR}{{R}_{o}}} \cdot {R}^{M - 1} & Eq . 4 \\ Γ ( M ) : Γ function & \\ {Ω}^{FT} ( q , R ) = \frac{4 π {R}^{3}}{{( q \cdot R )}^{3}} [ sin ( q \cdot R ) - ( q \cdot R ) \cdot cos ( q \cdot R ) ] & Eq . 5 \\ \begin{matrix}I ( q ) = \frac{8 {{π}^{2} ( 1 + \frac{4 {q}^{2} {R}_{o}^{2}}{{M}^{2}} )}^{- \frac{- 1 + M}{2}}}{( - 3 + M ) ( - 2 + M ) ( - 1 + M ) {q}^{6}} \\ \{ \begin{matrix}{M}^{3} ( 1 + \frac{4 {q}^{2} {R}_{o}^{2}}{{M}^{2}} ) [ {( 1 + \frac{4 {q}^{2} {R}_{o}^{2}}{{M}^{2}} )}^{\frac{- 3 + M}{2}} - \\ cos [ ( - 3 + M ) {tan}^{- 1} ( \frac{2 {qR}_{o}}{M} ) ] ] + \\ ( - 3 + M ) ( - 2 + M ) M \cdot {q}^{2} \cdot {R}_{o}^{2} [ {( 1 + \frac{4 {q}^{2} {R}_{o}^{2}}{{M}^{2}} )}^{\frac{- 1 + M}{2}} + \\ cos [ ( - 1 + M ) {tan}^{- 1} ( \frac{2 {qR}_{o}}{M} ) ] ] - \\ 2 ( - 3 + M ) {M}^{2} \cdot q \cdot {{R}_{o} ( 1 + \frac{4 {qR}_{o}^{2}}{{M}^{2}} )}^{\frac{1}{2}} sin [ ( - 2 + \\ M ) {tan}^{- 1} ( \frac{2 {qR}_{o}}{M} ) ]\end{matrix} \}\end{matrix} & Eq . 6\end{matrix}$$

The above-mentioned Eq.4 expresses gamma distribution as particle diameter distribution and of course, needless to say, it is permissible to use a particle diameter distribution function expressing particle diameter distribution other than the gamma distribution (for example, Gaussian distribution and the like). Any distribution is desired to be selected in order to realize high precision fitting between the simulated scattering curve and the actually measured scattering curve.

Next, the scattering function I(q) using the spherical model can be given as Eq.7, for example. In this case, the parameter [D, a] expressing the diameter and aspect ratio of the particle-like matter modeled according to the cylindrical model serves as fitting parameter indicating the distribution state of the particle-like matter as well as the distribution shape parameter [M]. The scattering function I(q) of the Eq. 7 in a function which expresses the X-ray scattering curve affected by various distribution states by selecting the value for [D, a, M] arbitrarily.

$$\begin{matrix}I ( q ) = \frac{2 {π}^{2} {ρ}_{o}}{{q}^{6}} {( \frac{{qD}_{o}}{2} )}^{3} \cdot \frac{{( \frac{M}{{qD}_{o}} )}^{M}}{Γ ( M )} \cdot {\int }_{0}^{∞} ⅆ x \cdot {x}^{M + 2} F ( a , x ) {ⅇ}^{- \frac{M}{{qD}_{o}} x} \\ F ( a , qD ) = {\int }_{0}^{π} sin θ ⅆ θ {\vert \frac{sin ( \frac{a \cdot qD}{2} \cdot cos θ ) {J}_{1} ( \frac{qD}{2} \cdot sin θ )}{{( \frac{qD}{2} )}^{2} sin θ cos θ} \vert}^{2}\end{matrix} \qquad \text{Eq. 7}$$

D:Diameter parameter

a:Aspect ratio parameter

M:Distribution size parameter

q:Scattering vector

Γ(M):Γfunction

Jₙ(z):Bessel function

The scattering vector used in the above-described respective equations takes into account the effect or refraction by the particle-like matter. In a thin film sample, the effect of refraction of incident X-ray on its surface affects the measured scattering curve seriously and simulation taking into account the effect of refraction is necessary for achieving high-precision non-uniform-density analysis. According to the invention of this application, a scattering function optimum for simulation is obtained by using scattering vector q taking into account the effect of refraction as given by the equation 2, accurately. More specifically, generally, although the scattering vector is q=(4πsinθs)/λ, in case of thin film, it is considered that there is a relationship of

$$2 {θ}_{s} = \sqrt{{θ}_{out} - 2 δ} + \sqrt{{θ}_{i n} - 2 δ} \qquad Eq . 8$$

among the scattering angle 2θs of the X-ray scattering by the particle-like matter, θin and θout and thus, this is introduced into a general equation. The critical angle θc obtained from the X-ray reflection curve is utilized in this scattering vector q (θc=√2δ).

The scattering function, which selectively uses any of Eqs. 3 to 6 and 7. simulated various kinds of scattering curves based on the average particle radius parameter Ro as the fitting parameter, distribution shape parameter M, diameter parameter D and aspect ratio parameter a, considering an influence by the particle-like matter strictly. Therefore, by optimizing the value of respective parameter [Ro, M] or [D, a, M] as described later, a simulated scattering curve, which agrees with the actually measured scattering curve, can be computed.

In Eq. 2, it is natural to consider the structure element of atom which constitutes the particle-like matter.

In Eqs.2 to 7, strictly speaking, not the scattering vector q but also its magnitude |q| is used. This is because although generally, it is handled as vector q, in the each of the above-described equations, it is assumed that the particle-like matter has random orientation and thus isotropy (not dependent of orientation) is assumed.

Computation on the simulated X-ray scattering curve by the above-described scattering function will be described further. First, after the same condition as at the time of actual measurement of the scattering curve is set up, if the scattering function (Eqs.3 to6) based on the spherical model is selected, the values of the average particle radius parameter Ro and distribution shape parameter M are selected arbitrarily and if a scattering function (Eq.7) based on the cylindrical model is selected, the values of the diameter parameter D, aspect ratio parameter a and distribution shape parameter M are selected arbitrarily. Then, by employing the Eq.8, an X-ray scattering curve when a selection value [Ro, M] or [D, a, M] under the condition for scanning θout with θin=θout±δω constant or scanning θin with θout constant is obtained.

More specifically, various parameters necessary for this, computation are Ro, M, D, a, q, θin, θout, δ, λ, ρo as evident from the above-described Eqs.2 to 7. of these parameters, δ, ρo are obtained from reflectivity curve, q can be computed from θin, θout, δ, λ and Ro, M, D, a are fitting parameters. Therefore, in simulation, only if the reflectivity curve is measured, computing the scattering function enables simulated X-ray scattering curve to be obtained easily in a short time.

It has been already described that the distribution of the particle-like matter affects the scattering curve obtained from the non-uniform-density sample seriously. The scattering function of the equation 2 takes into account that influence by the scattering vector or non-uniform-density scattering form factor and has achieved acquisition of high precision simulated scattering curve. However, the influence by the particle-like matter is diversified in various ways and for example, the refractive index, absorption effect and irradiation area of the X-ray entering into a sample are affected also. The correlation state between the particle-like matters is also a factor which affects the scattering curve.

Thus, according to the invention of this application, it is permissible to achieve a further precision fitting by considering these various influences by the non-uniform-density sample and introduce “absorption/irradiation area correction considering refraction and the like” (hereinafter referred to as absorption/irradiation area correction) or “particle-like matter correlation function” into the above-described scattering function. In this case, the scattering function can be given by the following equation, for example.

$$\begin{matrix}I ( {θ}_{i n} , {θ}_{out} ) = A \cdot I ( q ) \cdot S ( q ) & \\ q = \frac{4 π}{λ} sin [ \frac{\sqrt{{θ}_{i n}^{2} - 2 δ} + \sqrt{{θ}_{out}^{2} - 2 δ}}{2} ] & Eq . 9 \\ \begin{matrix}I ( {θ}_{i n} , {θ}_{out} ) & : Scattering function \\ q = \vert q \vert & : Magnitude of scattering vector \\ q & : Scattering vector \\ {θ}_{c} = \sqrt{2 δ} & : Critical angle \\ n = 1 - δ & : Index of refraction \\ λ & : X \text{-} ray wavelength\end{matrix} & \end{matrix}$$

In this scattering function, A is absorption/irradiation area correction and S(q) is particle-like matter correlation function. Of course, in this case also, it is permissible to select one based on the above-described spherical model or cylindrical model under I(q).

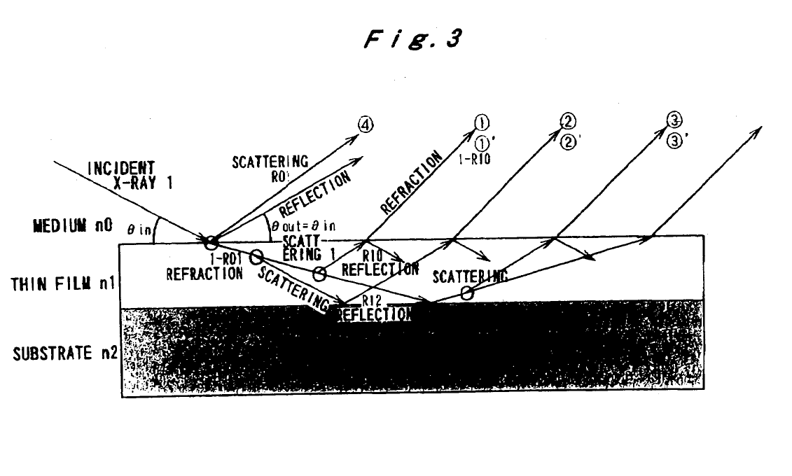

First, the absorption/irradiation area correction A will be described. FIG. 3 shows the state of X-ray in the non-uniform-density thin film (refractive index n1) formed on the substrate (refractive index n2). As exemplified in FIG. 3, in the non-uniform-density thin film containing the particle-like matter, it can be considered that there are, as X-rays emitted from the film surface, \mathring{1} an X-ray which after scattered by the particle-like matter in the film in the direction to the film surface, is refracted by the film surface to some extent and then emitted \mathring{2} an X-ray which after scattered by the particle-like matter in the film in the direction to an interface with the substrate, is reflected by the interface in the direction to the film surface, refracted by the film surface to some extent and then emitted and \mathring{3} an X-ray which is reflected at the interface in the direction to the film surface and scatted by the particle-like matter before reaching the film surface and refracted by the film surface to some extent and then emitted. In \mathring{1} to \mathring{3}, in some case, part thereof is reflected and returned into the film by the film surface while the remainder is emitted out of the film surface (\mathring{1}′, \mathring{2}′, \mathring{3}′).

Therefore, by introducing the absorption/irradiation area correction A considering refraction/reflection/scattering state of the X-ray in \mathring{1} to \mathring{3} and \mathring{1}′ to \mathring{3}′, a scattering function considering the particle-like matter in the sample with thin film further accurately can be achieved.

Absorption/irradiation area correction A₁ considering \mathring{1} can be given by Eq.10, for example.

$$\begin{matrix}{A}_{1} = \frac{d}{sin {θ}_{i n}} \cdot \frac{1 - {ⅇ}^{- ( \frac{1}{sin {θ}_{i n}^{′}} + \frac{1}{sin {θ}_{out}^{′}} ) μ d}}{( \frac{1}{sin {θ}_{i n}^{′}} + \frac{1}{sin {θ}_{out}^{′}} ) μ d} \\ {θ}_{i n}^{′} = \sqrt{{θ}_{i n}^{2} - 2 δ} \\ {θ}_{out}^{′} = \sqrt{{θ}_{out}^{2} - 2 δ} \\ μ : Absorption coefficient \\ d : Thickness of thin film\end{matrix} \qquad Eq . 10$$

In this absorption/irradiation area correction A₁, θin′=√(θᵢₙ²·2δ), d/sin θin, and (1·e^{.***})/(***) are considered for refraction correction, irradiation area correction and absorption effect correction, respectively (*** indicates (1/sinθ′ᵢₙ+1/sinθ′ₒᵤₜ) corresponding in Eq.10)

The absorption/irradiation area correction A₁ considering \mathring{1} can be given by Eq.11, for example.

A_{1′}=A₁

$${A}_{{1}^{′}} = {A}_{1} \cdot \{ ( 1 - {R}_{01} ( {θ}_{i n} ) ) \cdot \frac{{θ}_{i n}}{\sqrt{{θ}_{i n}^{2} - {θ}_{c}^{2}}} \cdot \frac{{θ}_{out}}{\sqrt{{θ}_{out}^{2} - {θ}_{c}^{2}}} \cdot ( 1 - {R}_{10} ( {θ}_{out} ) ) \} \qquad Eq . 11$$

The absorption/irradiation area correction A₂ considering \mathring{2} can be give by Eq.12, for example.

$${A}_{2} = \frac{d}{sin {θ}_{i n}} \cdot {ⅇ}^{- \frac{2 μ d}{sin {θ}_{out}^{′}}} \cdot {R}_{12} ( {θ}_{out}^{′} ) \qquad Eq . 12$$

The absorption/irradiation area correction A₂ considering \mathring{2}′ can be given by Eq.13, for example.

A_{2′}=A₂

$${A}_{{2}^{′}} = {A}_{2} \cdot \{ ( 1 - {R}_{01} ) \cdot \frac{{θ}_{out}}{\sqrt{{θ}_{out}^{2} - {θ}_{c}^{2}}} \cdot \frac{{θ}_{i n}}{\sqrt{{θ}_{i n}^{2} - {θ}_{c}^{2}}} \cdot ( 1 - {R}_{10} ) \} \qquad Eq . 13$$

The absorption/irradiation area correction A₃ considering \mathring{3} can be given by Eq.14, for example.

$${A}_{3} = \frac{d}{sin {θ}_{i n}} \cdot {ⅇ}^{- \frac{2 μ d}{sin {θ}_{i n}^{′}}} \cdot {R}_{12} ( {θ}_{i n}^{′} ) \qquad Eq . 14$$

The absorption/irradiation area correction A_{3′} considering \mathring{3}′ can be given by Eq.15, for example.

A_{3′}=A₃

$${A}_{{3}^{′}} = {A}_{3} \cdot \{ ( 1 - {R}_{01} ) \cdot \frac{{θ}_{out}}{\sqrt{{θ}_{out}^{2} - {θ}_{c}^{2}}} \cdot \frac{{θ}_{i n}}{\sqrt{{θ}_{i n}^{2} - {θ}_{c}^{2}}} \cdot ( 1 - {R}_{10} ) \} \qquad Eq . 15$$

And in Eqs.12 to 15, q is given by Eq.16.

$$q = \frac{4 π}{λ} sin [ \frac{\sqrt{{θ}_{i n}^{2} - 2 δ} - \sqrt{{θ}_{out}^{2} - 2 δ}}{2} ] \qquad Eq . 16$$

The above-described eqns.10 to 15 may be employed as the absorption/irradiation area correction A in Eq.9. Eqs.10 to 15 can be used in combination corresponding to the thin film of an object. Eq.17 is an example thereof while its upper row considers Eqs. 10, 12, 14 and its lower row considers Eqs. 11, 13, 15.

I(θᵢₙ,θₒᵤₜ)=A₁·I(q)·S(q)+A₂·I(q)·S(q)+A₃·I(q)·S(q)

I(θᵢₙ,θₒᵤₜ)=A_{1′}·I(q)·S(q)+A_{2′}·I(q)·S(q)+A_{3′}·I(q)·S(q) Eq. 17

Further, because naturally, the X-ray is scattered on the surface of the film, correction may be carried out for the scattered X-ray (\mathring{4} in FIG. 2). This correction may be carried out according to a well known equation (for example, S. K. sinha, E. B. Sirota, and G.Garoff, “X-ray and neutron scattering from rough surfaces”, Physical Review B, vol.38, no.4,pp.2297-2311, August 1988, Eq(4. 41)).

Because of the above-described \mathring{1} to \mathring{4}, \mathring{1} can be generated in the bulk body also, the absorption/irradiation area correction based on Eq.10 can be used for analyzing of the non-uniform-density bulk body so as to improve analysis accuracy. In this case, the thickness d in Eq.10 is thickness d of the bulk body.

Next, particle-like matter correlation function S(q) will be described and this is a function indicating the correlation between the particle-like matters and for example, a following equation can be an example thereof.

S(q)=1+∫dr(n(r)−nₒ)e^{iqr} Eq. 18

n(r):Density distribution function of particle-like matter

n₀:Average number density of particle-like matter

q:Scattering vector

r:Spatial coordinate

In an actual simulation, it is necessary to use an appropriate specific model capable of expressing distribution state an density distribution function n (r) of the particle-like matter as the particle-like matter correlation function S(q) given in the form of the Eq.18.

For example, estimating that the particle-like matters are distributed under the nearest distance L and correlation function η as an example of the specific model, these L and η are regarded as a fitting parameter. The particle-like matter correlation function S(q) of this case can be given as the following equation, for example.

$$\begin{matrix}S ( q ) = \frac{1}{1 - C ( q )} \\ C ( q ) = \frac{24 η}{{( 1 - η )}^{4} {( q \cdot L )}^{3}} [ \begin{matrix}{( 1 + 2 η )}^{2} ( \begin{matrix}sin ( q \cdot L ) - \\ q \cdot L cos ( q \cdot L )\end{matrix} ) - \\ 6 {η ( 1 + \frac{η}{2} )}^{2} ( \begin{matrix}2 sin ( q \cdot L ) - \\ q \cdot L cos ( q \cdot L ) - \\ \frac{2 ( 1 - cos ( q \cdot L ) )}{q \cdot L}\end{matrix} ) + \\ \frac{1}{2} {η ( 1 + 2 η )}^{2} \{ \begin{matrix}( 4 - \frac{24}{{( q \cdot L )}^{2}} ) sin ( q \cdot L ) - \\ ( q \cdot L - \frac{12}{q \cdot L} ) cos ( q \cdot L ) + \\ \frac{24 ( 1 - cos ( q \cdot L ) )}{{( q \cdot L )}^{3}}\end{matrix} \}\end{matrix} ]\end{matrix} \qquad Eq . 19$$

L:Inter-particle nearest distance parameter

η:Inter-particle correlation coefficient (packing density) parameter

In case of scattering function of Eq.9 incorporating the particle-like matter correlation function of Eq.19, various parameters necessary for computing of the simulated X-ray scattering curve are Ro, M, a, M, q(θin, θout, λ, δ) ρo, μ, d, L, η. Although the parameters which multiply after the equation 2 described above are μ, d, L, η, μ and d can be determined from a non-uniform-density sample used for measurement. The L and η are fitting parameters for carrying out fitting between the simulated scattering curve and actually measured scattering curve like Ro, M, D, a, they indicate the nearest distance between particle-like matters and correlation coefficient. Therefore, more X-ray scattering curves can be simulated easily only by measuring the X-ray reflectivity curve and then adjusting values of average particle radius parameter Ro, distribution shape parameter M, diameter parameter D, aspect ratio parameter a, inter-particle nearest distance parameter L and inter-particle correlation coefficient parameter η.

Although introduction processes for the above-described scattering function, non-uniform-density scattering form factor, particle diameter distribution function, absorption/irradiation area correction item and inter-particle correlation function are omitted here because they are each comprised of multiple steps, a feature of the invention of this application is using a scattering function for simulating the X-ray scattering curve according to various kinds of the fitting parameters and if each of the above-described equations are calculated, a simulation X-ray scattering curve necessary for non-uniform-density analysis can be obtained.

Basically, each of the above-described equations (Eqs. 2 to 19) can be obtained by developing the well known basic scattering function given by the following Eq.20 by using Eqs.21 and 22 considering the non-uniform distribution of the particle-like matter.

$$\frac{ⅆ σ}{ⅆ Ω} = \int {ρ ( r )}^{′} {ⅇ}^{- ⅈ {qr}^{′}} ⅆ {r}^{′} \int ρ ( r ) {ⅇ}^{ⅈ qr} ⅆ r \qquad Eq . 20$$

ρ(r):Electronic density distribution in non-uniform-density sample accompanied by distribution of particle-like matter

q:Scattering vector

r:Spatial coordinate

$$ρ ( r ) = {\sum }_{i} {ρ}_{i} ( r - {R}_{i} ) \qquad Eq . 21$$

Rᵢ:Position of particle-like matter i

ρᵢ(r−Rᵢ):Electronic density distribution of particle-like matter i

$$\begin{matrix}{Ω}^{FT} ( q ) = \int ⅆ r ⟨ ρ ( r ) ⟩ {ⅇ}^{ⅈ qr} \\ ⟨ ρ ( r ) ⟩ = \frac{{\sum }_{i} {ρ}_{i} ( r )}{N}\end{matrix} \qquad Eq . 22$$

Ω^{FT}(q):Particular form factor

<ρ(r)>:Average electronic density distribution of particle-like matter

N:Quantity of particle-like matter

N (quantity of particle-like matter) in Eq.22 can be obtained from analyzing object area of the non-uniform-density sample by using the following equation.

$$\begin{matrix}N = \frac{{S}_{o} d}{sin {θ}_{i n}} \cdot \frac{1 - {ⅇ}^{- ( \frac{1}{sin {θ}_{i n}^{′}} + \frac{1}{sin {θ}_{out}^{′}} ) μ d}}{μ d ( \frac{1}{sin {θ}_{i n}^{′}} + \frac{1}{sin {θ}_{out}^{′}} )} \cdot \frac{1}{{R}_{o}^{3}} \\ {S}_{o} = {L}_{x} {L}_{y}\end{matrix} \qquad Eq . 23$$

Lₓ:Inter-particle nearest distance in x direction

L_{y}:Inter-particle nearest distance in y direction

d:Thickness of sample

Of course, the above-mentioned equations are only an example and needless to say, the variable names and arrangement used therein are not restricted to the above-mentioned ones.

Although the scattering functions of Eqs.3, 7 and 9 utilize [Ro, M], [D, a, M], [L, η] as the fitting parameter, it is permissible to use a scattering function expressing the X-ray scattering curve according to a fitting parameter indicating the content ratio of the particle-like matter and correlation distance. In this case, the scattering function can be given by the following Eqs.24 and 25.

$$\begin{matrix}I ( {θ}_{i n} , {θ}_{out} ) = {\vert {Ω}^{FT} ( q ) \vert}^{2} \\ q = \frac{4 π}{λ} sin [ \frac{\sqrt{{θ}_{i n}^{2} - 2 δ} + \sqrt{{θ}_{out}^{2} - 2 δ}}{2} ]\end{matrix} \qquad Eq . 24$$

I(θᵢₙ, θₒᵤₜ):Scattering function

Ω^{FT}(q):Non-uniform-density scattering form factor

q=|q|:Magnitute of scattering vector

q:Scattering vector

θ_{c}=\sqrt{2δ}:Critical angle

n=1−δ:Indext of refraction

λ:X-ray wavelength

$${Ω}^{FT} ( q ) = {( Δ ρ )}^{2} \frac{8 π P ( 1 - P ) {ξ}^{3}}{{( 1 + {q}^{2} {ξ}^{2} )}^{2}} \qquad Eq . 25$$

Δρ:Difference in density between particle-like matter and other sample composition matter

P:Volume fraction parameter of particle-like matter

ξ:Correlation distance parameter of particle-like matter

In case where the non-uniform-density sample is porous film an described later and the particle-like matter is of fine particles forming the porous film or pores (see the second embodiment), Δρ in Eq.24 is a difference in density between the fine particle or pore and other matter (not substrate but a matter constituting the film itself) constituting the porous film and P is fine particle ratio or pore ratio and ξ is a correlation distance between the fine particles or pores.

If this scattering function is used, fitting between the simulated X-ray scattering curve and actually measured scattering curve in carried out while changing the P and ξ as the fitting parameter.

Further, a following scattering function can be used. Although an ordinary X-ray diffraction meter is capable of measuring the direction of angle of response or rotation direction of goniometer with an excellent parallelism, it has a large scattering in the direction perpendicular to that. Because this affects the profile of small angle scattering, the slit length needs to be corrected. If this slit length correction is considered, when the slit function is set as W(s), a scattering function I_{obs} (q) to be measured with respect to the scattering function I(q) can be given by the following equation.

$${I}_{obs} ( q ) = {\int }_{- ∞}^{∞} I ( \sqrt{{q}^{2} + {s}^{2}} ) W ( s ) ⅆ s \qquad Eq . 26$$

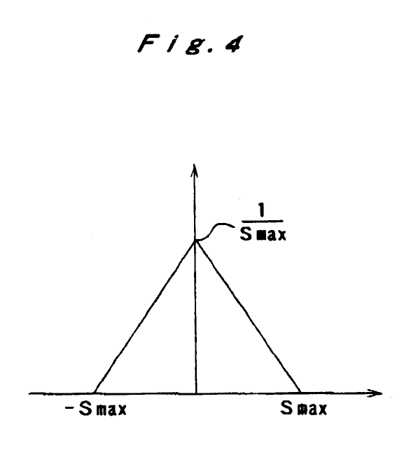

Therefore, the above-described respective scattering function I(q) may be replaced with the scattering function I_{obs}(q) of Eq.26. FIG. 4 is a diagram showing an example of slit function W(s). Of course, this is an example and the slit function W(s) may be selected appropriately to correspond to the X-ray diffraction meter.

<Step s5> After the simulated X-ray scattering curve is computed with the scattering function as described above, fitting between the simulated X-ray scattering curve and the actually measured X-rays scattering curve is carried out. In this fitting, the degree of coincidence of both the curves (or difference between both the curves) is considered. For example, the difference between both the curves can be obtained from this equation.

$${x}^{2} = {\sum }_{i} {( log {I}_{i} ( exp ) - log {I}_{i} ( cal ) )}^{2} \qquad Eq . 27$$

Iᵢ(exp):Actually measured data at measuring point i

Iᵢ(cal):Simulate data at measuring point i

<Step s6> If the degree of coincidence (or difference) is a predetermined value or within a predetermined range, it is determined that both the curves coincide with each other and otherwise, it is determined that both the curves do not coincide.

<Step s6 No→step s4→step s5> If it is determined than both the curves do not coincide, the fitting parameter indicating the distribution state of the particle-like matter in the scattering function is changed and again, the simulated X-ray scattering curve in computed and whether or not it agrees with the actually measured X-ray scattering curve is determined. This procedure is repeated by adjusting and changing the values of the fitting parameter until both the curves come to agree with each other. In case of a scattering function given by Eq.3 or 7, the value of [Ro, M] or [D, a] is changed. In case of a scattering function given by Eq.9 incorporating the particle-like matter correlation function of Eq.19, the values of [L, ρ] as well as [Ro, M] or [D, a] are changed and in case of a scattering function given by Eq.24, the value of [P, ξ] is changed.

<Step s6 Yes→e step s7> Then, the selection value of the fitting parameter when the simulated X-ray scattering curve agrees with the actually measured X-ray scattering curve becomes a value which indicates the distribution state of the particle-like matter in the non-uniform density sample of an analyzing object. The values of [Ro, M] are the average particle radius and distribution shape of the particle-like matter, the values of [D, z, M] are the diameter, aspect ratio and distribution shape of the particle-like matter, the values of [L, η] are the nearest distance between the particle-like matters and correlation coefficient and the values of [P, ξ] are the content ratio and correlation distance of the particle-like matter.

In this fitting, for example, by using non-linear least squares method, an optimum value of each fitting parameter can be obtained effectively.

Because each function considering the non-uniformity of density is utilized as described above, the degree of coincidence between the simulated X-ray scattering curve and the actually measured X-ray scattering curve is intensified considerably, so that each fitting parameter indicates the distribution state of actual particle-like matter very accurately. Therefore, the non-uniformity of densities of the thin film and bulk body can be achieved very highly accurately.

Further, because measurement for the non-uniform-density sample includes only measurement of reflectivity and measurement of scattering curve, measuring time does not take long or limitation of the kind of the thin film about whether or not gas can invade into thin film is not required unlike the conventional gas absorption method or it is not necessary to peel thin film formed on the substrate unlike the conventional small angle scattering method. Therefore, the non-uniform-density analysis can be achieved in a short time without destruction to various kinds of the non-uniform-density bulk body an well as various kinds of the non-uniform-density thin film.

Although the above description concerns the case where the X-ray in used, needless to say, the distribution state of the particle-like matter in the non-uniform-density sample and the average density of the non-uniform-density sample can be analyzed by using such particle beam as neutron beam, electron beam also. Further, the above-described respective scattering functions can be applied to the reflectivity curve and scattering curve of the particle beam as they are (the “X-ray” is replaced with “particle beam” when reading the respective scattering functions). Consequently, very accurate agreement between the simulated particle beam scattering curve and actually measured particle beam scattering curve is achieved, so that the non-uniformity of density can be analyzed at a high accuracy.

According to the non-uniform-density sample analyzing method of the invention of this application, computation steps for simulation and fitting are executed actually with a computer (general-purpose computer or analysis specialized computer).

Further, the non-uniform-density sample analyzing device provided by the invention of this application can be achieved in the form of for example, software status which make the general-purpose computer function, computer (analyzing device) dedicated for analysis and software (program) which is built in that device. Further, the non-uniform-density sample analyzing system of the invention of this application includes the X-ray/particle beam measuring device and various kinds of the non-uniform-density sample analyzing device, and both the apparatuses are so constructed as to be capable of receiving/transmitting bi-direction or single-direction data.

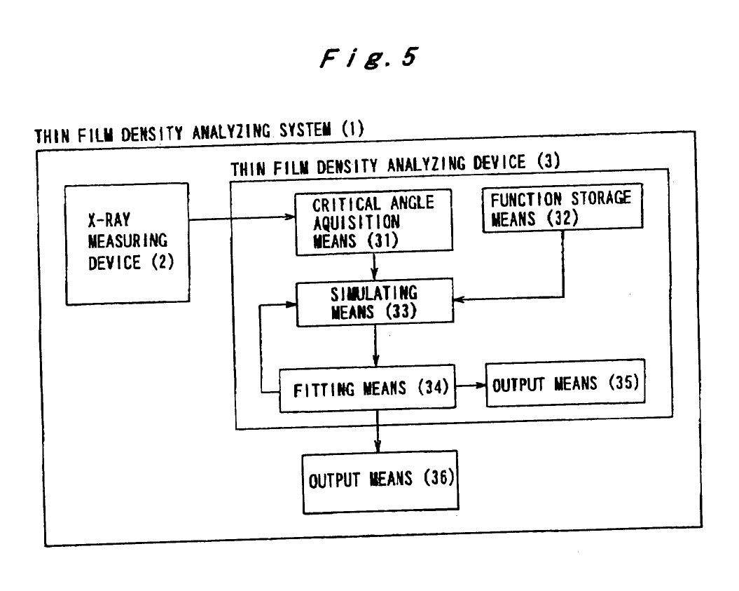

FIG. 5 is a block diagram showing an embodiment of the non-uniform-density sample analyzing system which executes the non-uniform-density sample analyzing method of the invention of this application in case of using the X-ray and analyzes the average particle diameter and distribution shape of the particle-like matter of the non-uniform-density sample.

The non-uniform-density sample analyzing system (1) shown in FIG. 5 comprises the X-ray measuring device (2) and the non-uniform-density sample analyzing device (3).

The X-ray measuring device (2) measures the X-ray reflectivity curve and X-ray scattering curve of the non-uniform-density sample. If the non-uniform-density sample is thin film sample, it is permissible to use a goniometer (usually, thin film sample in placed in its sample chamber) or the like and it is measured by setting up the X-ray incident angle θin, X-ray emission angle θout, and scattering angle 2θ=θin+θout and then scanning. Like described previously (see steps s1, s2 about analyzing method), measurement of reflectivity curve is carried out under θin=θout, the measurement of scattering curve is executed by scanning θout with θin=θout±Δω, θin constant or scanning θin with θout constant.

The non-uniform-density sample analyzing device (3) comprises the critical angle acquisition means (31), the function storage means (32), the simulating means (33) and the fitting means (34).

The critical angle acquisition means (31) introduces a critical angle θc from the measured X-ray reflectivity curve by the X-ray measuring device (2) and the actually measured X-ray scattering curve like described previously (see step s3). Further, it may be so constructed that δ can be computed from this critical angle θc.

Basically the function storage means (32) stores the above-described respective functions. The above-described other equations used for the respective scattering functions are stored therein.

The simulating means (33) selects the value of various kinds of the fitting parameters and computes the simulated X-ray scattering curve using the scattering function (including other necessary functions) from the function storage means (32) and θc (or δ) from the critical angle acquisition means (31), like described previously (see step s4).

The fitting means (34) executes fitting between the simulated X-ray scattering curve from the simulating means (33) and the actually measured X-ray scattering curve from the X-ray measuring device (2) like described previously (see step s5).

Data such as the measured X-ray reflectivity/scattering curve, θin/θout necessary for simulation and fitting is automatically transmitted from for example, the X-ray measuring device (2) to the non-uniform-density sample analyzing device (3), and preferably automatically transmitted to the critical angle acquisition means (31), the simulation means (33) and the fitting means (34) corresponding to each data. Of course, manual input is permissible.

If the above-described respective equations are used for computation of the simulated X-ray scattering curve, as described above, the simulating means (33) requires θin, θout, λ, μ, d, ρo as well an θc (or δ). For example, θin and θout (or 2θ) may be supplied by automatic transmission from the X-ray measuring device (2) while μ, λ, d, ρo may be supplied by manual input or from preliminary storage or computation elsewhere. The non-uniform-density sample analyzing system (1) or non-uniform-density sample analyzing device (3) require an input means, storage means, computation means and the like for it and needless to say, these various means and the simulating means (33) are so constructed as to be capable of transmitting/receiving data.

Like described previously (see steps s6, s7), the non-uniform-density sample analyzing device (3) repeats computation of the simulated X-ray scattering curve while changing various kinds of the fitting parameters by the simulating means (33) until the simulated X-ray scattering curve agrees with the actually measured X-ray scattering curve by means of the fitting means (34). If both the curves agree with each other, the value of the fitting parameter is analyzed as the distribution state of an actual particle-like matter.

In the example shown in FIG. 5, the non-uniform-density sample analyzing device (3) is provided with an output means (35) or the non-uniform-density sample analyzing system (1) is provided with an output means (36), so that the result of analysis (average particle diameter and distribution shape) is outputted through these output means (35), (36) such as display, printer, incorporated/separate storage means. Further, in order to reflect the analysis result by the non-uniform-density sample analyzing system (1) or the non-uniform-density sample analyzing device (3) to production of the thin film, the analysis result may be transmitted directly to the thin film producing device or its control device.

If the above-described non-uniform-density sample analyzing device (3) is achieved in the form of software which can be stored, started and operated by means of the general-purpose computer or analysis dedicated computer, the above-described respective means are achieved as programs for executing each function. Further, in case where it is an analysis dedicated computer (analyzing device) itself, the above-described respective means can be achieved as arithmetic logic circuit (including data input/out, storage functions) for executing each function. Then, in the non-uniform-density sample analyzing system (1), preferably, the non-uniform-density sample analyzing device (3) of each embodiment is so constructed as to be capable of transmitting/receiving data with the X-ray measuring device (2). In selecting an optimum value of the fitting parameter by the simulating means (33), completely automatic analysis with the computer is enabled by adding a function for automatically selecting according to the least squares method so that the degree of coincidence between the simulated curve and actually measured curve in raised (for example, approaches a predetermined value). Of course, arbitrary manual input enable in permitted.

The invention of this application has the above-described features. The examples are shown with reference to the accompanying drawings and then, the embodiments of the invention of this application will be described in detail.

### EXAMPLES

Example 1

A simulation of the X-ray scattering curve executed an an example of the invention of this application will be described below. In this simulation, a simulated X-ray scattering curve is computed using a scattering function in Eq.6 based on the spherical model as I(q).

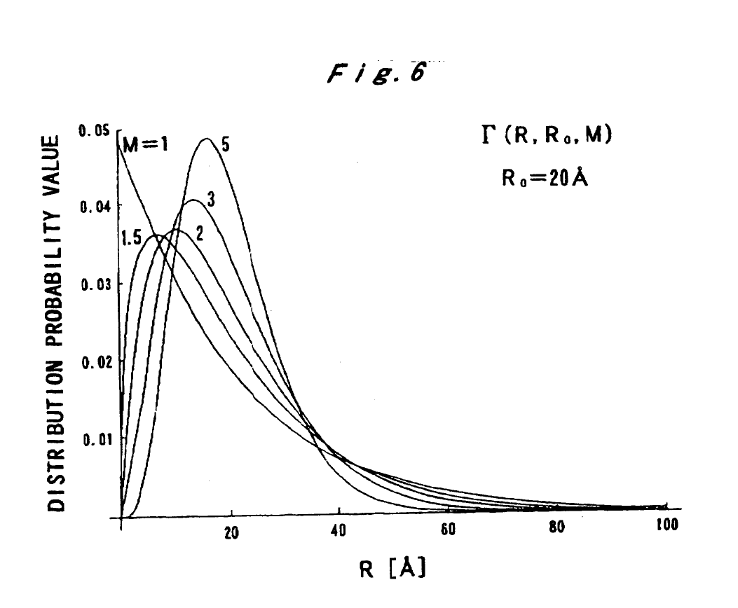

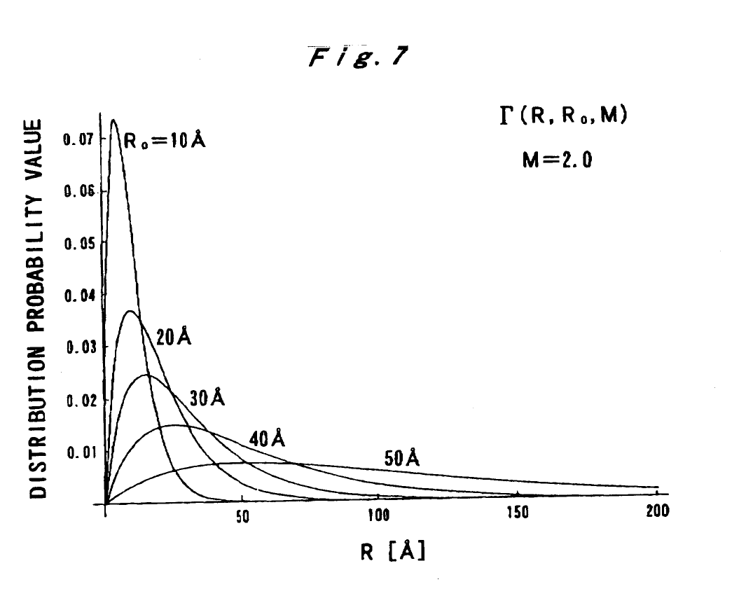

FIGS. 6 and 7 show examples of computation on gamma distribution of the average particle radius parameter Ro and distribution shape parameter. The gamma distribution shown in FIG. 6 indicates a case where M=1, 1.5, 2, 3, 5 is selected with Ro=20 [A] fixed, while the gamma distribution shown in FIG. 7 indicates a case where Ro=10, 20, 30, 40, 50 [A] is selected with M=2.0 fixed. Its abscissa axis indicates R[A] and its ordinate axis indicates distribution probability value. As evident from FIGS. 6 and 7, various types of particle diameter distributions can be obtained corresponding to the values of the average particle radius parameter Ro and the distribution shape parameter M.

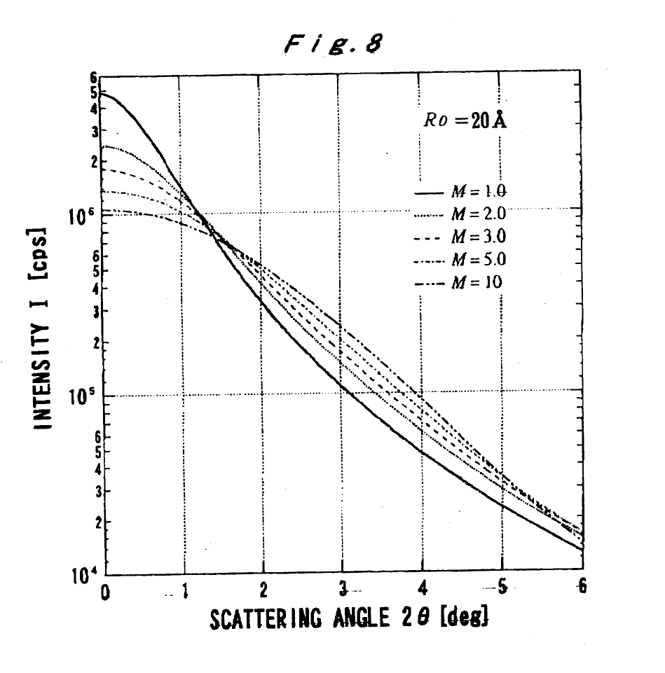

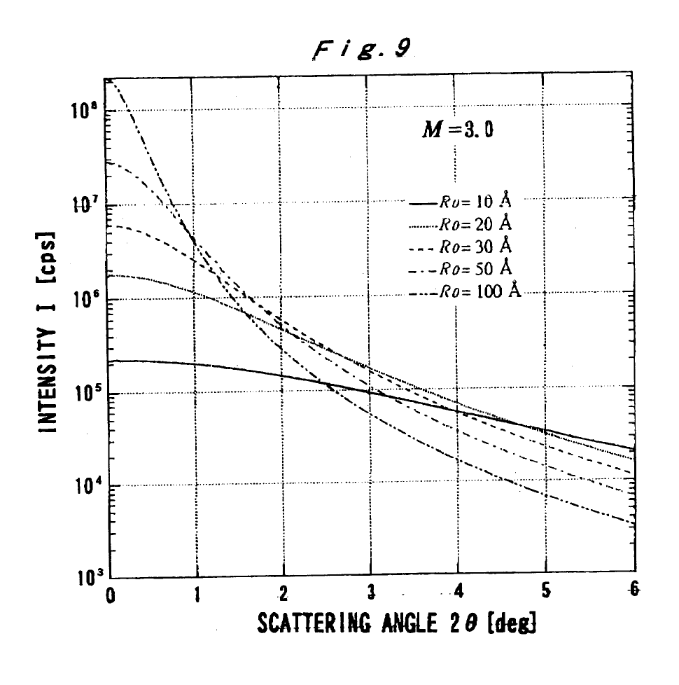

Next, FIGS. 8 and 9 show examples of simulated X-ray scattering curves computed by selecting still another [Ro, M]. Respective curves in FIG. 8 indicates cases where M=1.0, 2.0, 3.0, 5.0, 10 is selected with Ro=20 [A] fixed, while respective gamma distributions in FIG. 9 indicate cases where Ro=10, 20, 30, 50, 100 [A] is selected with M=3.0 fixes. The abscissa axis indicates a scattering angle 2θ [deg], while the ordinate axis indicates X-ray intensity I[cps]. It is assumed that λ=1.54 Å.

As evident from FIGS. 8 and 9, various types of the simulated X-ray scattering curves can be computed corresponding to respective gamma distributions. Therefore, high-accuracy analysis on the average particle diameter and distribution shape can be realized by only carrying out the measurement of the X-ray reflectivity curve and X-ray scattering curve and the fitting between them while adjusting the average particle radius parameter Ro and distribution shape parameter M. Analysis on the order of several nanometer can also be achieved.

Of course, even when simulation is carried out using Eq.7 based on the cylindrical model as I(q), the non-uniformity of density of various non-uniform-density samples can be analyzed at a high freedom and accuracy depending on the diameter parameter D, aspect ratio parameter a and distribution shape parameter M.

Example 2

Here, a porous film, which was thin film sample, was prepared actually as a non-uniform-density sample and then, the distribution state of the pores forming the porous film of the invention of this application was analyzed. Thus, its result will be described here.

Recently, in order to suppress operation delay induced by increase of interlayer capacity with intensified integration of the semiconductor integration circuit, demand for reduction of dielectric constant in the interlayer insulation film has been increased considerably. To promote the reduction of the dielectric constant, a number of films having fine particles or pores as the interlayer insulation film have been researched and developed. This film is the porous film. The porous film has a very low dielectric constant originated from the distribution of the pores and is very useful for high integration of the semiconductor. The porous film is divided to closed porous film in which a great number of fine particles or pores are dispersed in inorganic thin film or organic thin film and open porous film in which gap between the fine particles dispersed in the form of a substrate acts as the pore.

In this example, the porous film in which the pores are dispersed in SiO₂ film on the Si substrate was prepared. Further, the measuring condition for the X-ray scattering curve was set to θin=θout±0.1. When computing the simulated X-ray scattering curve, the scattering function in Eq.9 incorporating the Eq.6 by the spherical model was used as I(q), the one indicating the gamma distribution in Eq.5 was used as the particle diameter distribution function, the one given in the form of the Eq.10 was used as the absorption/irradiation area correction A and the one given by Eq. 19 was used an the particle-like matter correlation function S(q).

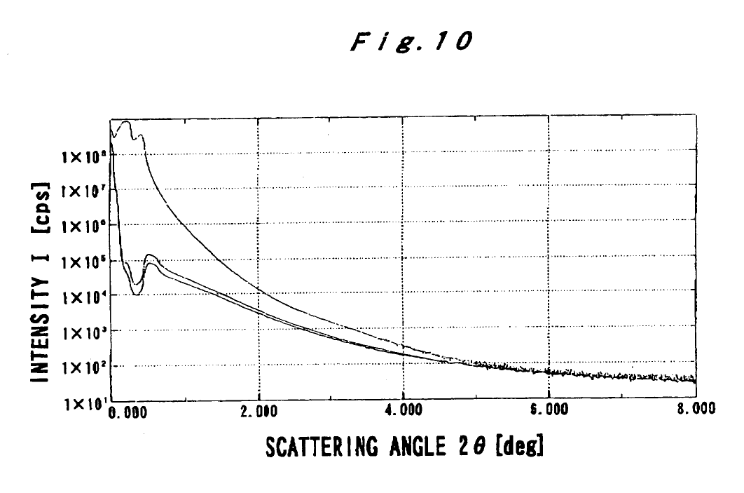

First, FIG. 10 shows measuring results of the X-ray reflectivity curve and X-ray scattering curve. The abscissa axis indicates 2θ/ω[^{.}] while the ordinate axis indicates the intensity I[cps]. Because in the X-ray reflectivity curve indicated in this FIG. 10, the angle in which the X-ray intensity drops rapidly is about 0.138°, this was regarded as the critical angle θc. Of course, determination of this critical angle θc can be executed with the computer.

Respective parameter values necessary for the scattering function of Eq.9 are as follows.

2θ=0°˜8°

ρ=0.91 g/cm³

δ=2.9156×10⁻⁶

μ=30 cm⁻¹

d=4200 Å

λ=1.5418 Å

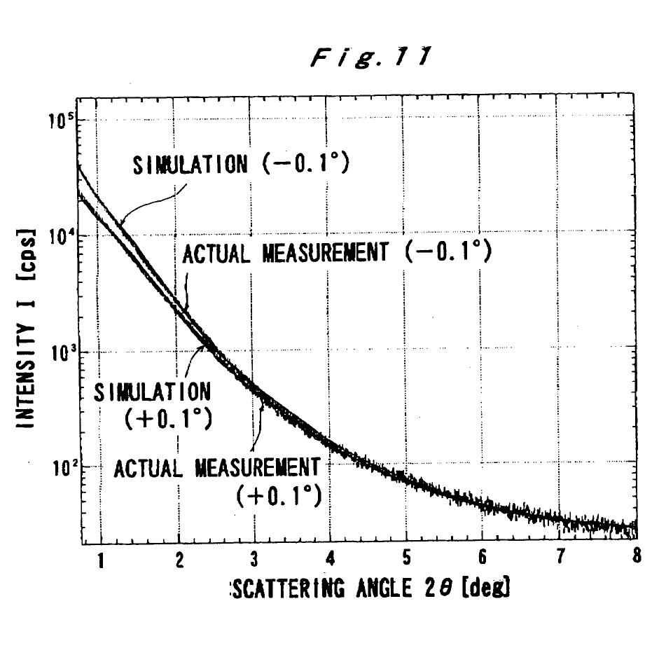

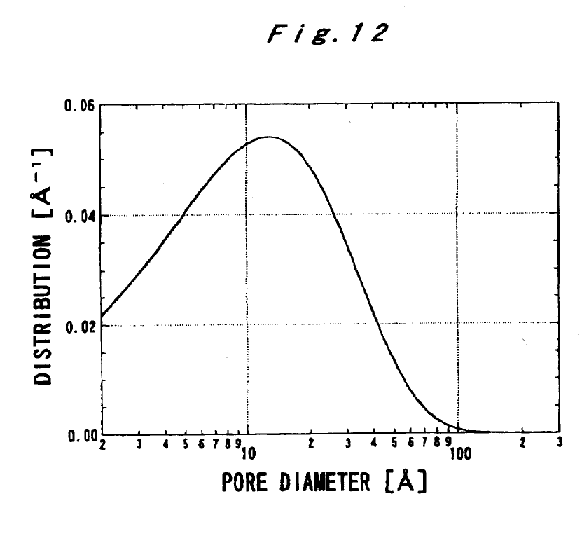

FIG. 11 shows the computed simulation X-ray scattering curve at δω=0±0.1° and the actually measured X-ray scattering curve in the overlay condition. As apparent from this FIG. 11, the both curves indicate a very high coincidence. At this time, the optimum values of the average particle radius parameter Ro and the distribution shape parameter M are Ro=10.5 Å and M=2.5 and the optimum values of the nearest distance parameter L and the correlation coefficient parameter η are L=30 Å and η=0.6. Therefore, it can be regarded that these respective values are average particle diameter of the pore in actual porous film, distribution shape, nearest distance and correlation coefficient. FIG. 12 indicates the distribution of the pore size obtained in this way.

Example 3

Here, about the porous film in which the pores are distributed in the SiO₂ thin film on the Si substrate, the simulated X-ray scattering curve was computed according to the scattering function (case A) in Eq.9 incorporating Eq.10 as the absorption/irradiation area correction A and the scattering function (case B) in Eq.17 lower row incorporating Eqs.10 to 15 all at once as the absorption/irradiation area correction A and then, the degree of coincidence with the actually measured scattering curve was compared. Both the cases A and B use the one based on the spherical model in Eq.6 I(q) , the one expressing the gamma distribution in Eq.5 as the particle diameter distribution function and the one based on Eq.19 as the particle-like matter correlation function S(q). Further, the measuring condition of the X-ray scattering curve was set to θin=θout−0.1°.

Respective parameters necessary for computation are as follows.

θc=0.145°

2θ=0˜4°

ρ=0.98 g/cm³

δ=3.17×10⁻⁶

μ=33.7 cm⁻¹

d=6000 Å

λ=1.5418 Å

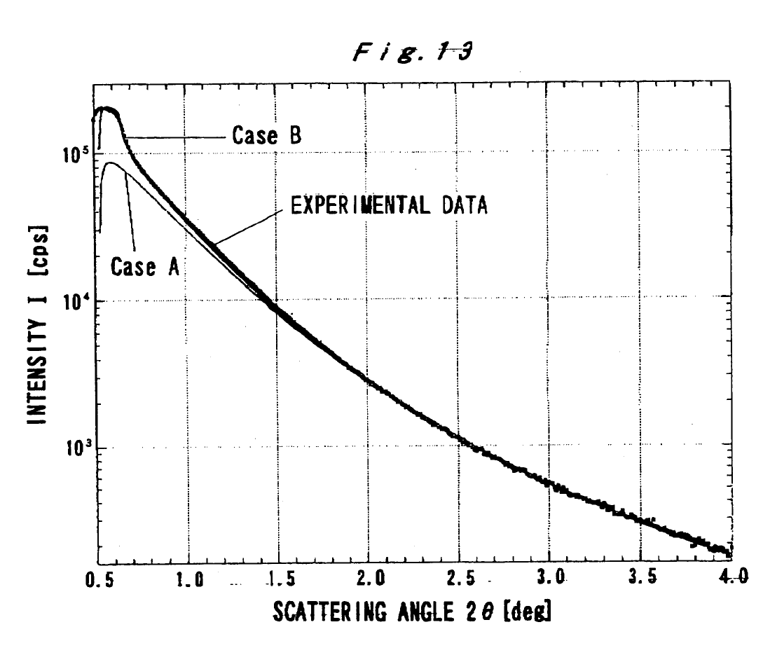

FIG. 13 shows the respective simulated X-ray scattering curves and the actually measured scattering curve in the overlay condition. As evident from FIG. 13, although a small crest is formed on a first portion of an actually measured curve, that crest is simulated more accurately in case A than case B. Therefore, by summing up all the equations 10-15 rather than correcting the scattering function according to only Eq.10, that is, considering all \mathring{1}, \mathring{1}′, \mathring{2}, \mathring{2}′, \mathring{3}, \mathring{3}′ in the above-described FIG. 3, more accurate fitting adaptive for various types of refraction, reflection and scattering of the X-ray by the particle-like matter is achieved so as to improve analysis accuracy. In the meantime, the optimum values of the average particle radius parameter Ro and the distribution shape M were Ro=18.5 and M=1.9.

Of course, \mathring{1}, \mathring{1}′, \mathring{2}, \mathring{2}′, \mathring{3}, \mathring{3}′, \mathring{4} in FIG. 3 can be selected in any combination corresponding to a sample of analysis object, so that simulation having a higher freedom is enabled, thereby the accuracy being improved further.

Example 4

Here, fitting between the simulated X-ray scattering curve and the actually measured X-ray scattering curve was tried using the scattering function of Eq.9 incorporating Eq.7 based on the cylindrical model as I(q). Respective various parameter values are as follows.

θin=θout−0.1°

θc=0.145°

2θ=0˜8°

ρ=0.98 g/cm³

δ=3.17×10⁻⁶

μ=33.7 cm⁻¹

d=3800 Å

λ=1.5418 Å

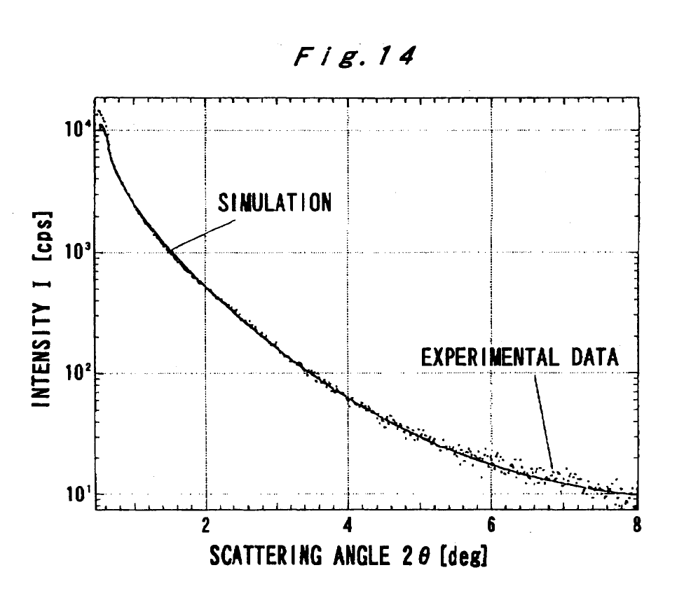

FIG. 14 shows respective simulated X-ray scattering curve and actually measured X-ray scattering curve in overlay condition. An evident from FIG. 14, this simulated curve has a very high degree of coincidence with the actually measured curve. Therefore, even when the distribution state is simulated by modeling the pores according to the cylindrical model, accurate analysis upon the non-uniformity of the density is achieved about the porous film used in this example. At this time, the diameter parameter D is 11A, the aspect ration parameter a is 2 and the distribution shape parameter M is 2.9.

As evident from the examples 3 to 5, the invention of this application enables very accurate distribution state on the porous film to be analyzed on the order of nanometer. Of course, in case where the scattering function of the Eq. 24 is employed, the porosity ratio P and the correlation distance ξ can be analyzed accurately if this model is appropriate.

Of course, needless to say, high fitting degree can be achieved for various types of thin films or bulk body as well as the porous film, so that excellent analysis upon the non-uniformity of density is enabled.

Although the above-described respective examples concern cases where the X-ray is employed, of course, highly accurate analysis can be achieved also even if such particle beam as electron beam, neutron beam is used. In this case, in the non-uniform-density sample analyzing system (1) exemplified in FIG. 5, the X-ray measuring device (2) acts as a particle beam measuring device so as to measure a particle beam reflectivity curve and a particle beam scattering curve. The various means (31), (33), (34) in the non-uniform-density sample analyzing device (3) introduces a critical angle and the like from the particle beam reflectivity curve so as to compute a simulated particle beam scattering curve and execute fitting between the simulated particle beam scattering curve and the actually measured particle beam scattering curve. Because the above-described function equations can be applied to the particle beam, the same function storage means (32) may be employed.

The invention of this application is not restricted to the above-described examples, however, it in needless to say that other various examples can be achieved about its detail.

Industrial Applicability

As described in detail, according to the non-uniform-density sample analyzing method, the non-uniform-density sample analyzing device and the non-uniform-density sample analyzing system of the invention of this application, the average density of the thin film or the bulk body as well an the distribution state (average particle diameter, distribution shape, nearest distance, correlation coefficient, content ratio, correlation distance and the like) of the particle-like matter in the thin film or bulk body can be analyzed at a high accuracy in a short time without any destruction. Further, the thin film and bulk body in which the average density and non-uniformity of density are taken into account objectively and accurately can be achieved.

1. An non-uniform-density sample analyzing method for analyzing distribution state of particle-like matter in a non-uniform-density sample, comprising:
computing a simulated X-ray scattering curve under the same condition as a measuring condition of an actually measured X-ray scattering curve by using a scattering function expressing a X-ray scattering curve according to a fitting parameter indicating distribution state of particle-like matter; and
carrying out fitting between the simulated X-ray scattering curve and the actually measured X-ray scattering curve while changing the fitting parameter,
wherein the value of the fitting parameter when the simulated X-ray scattering curve agrees with the actually measured X-ray scattering curve serves to indicate the distribution state of the particle-like matter in the non-uniform-density sample.

2. An non-uniform-density sample analyzing method for analyzing distribution state of particle-like matter in a non-uniform-density sample, comprising:
computing a simulated particle beam scattering curve under the same condition as a measuring condition of an actually measured particle beam scattering curve by using a scattering function expressing a particle beam scattering curve according to a fitting parameter indicating distribution state of particle-like matter; and
carrying out fitting between the simulated particle beam scattering curve and the actually measured particle beam scattering curve while changing the fitting parameter,
wherein the value of the fitting parameter when the simulated particle beam scattering curve agrees with the actually measured particle beam scattering curve serves to indicate the distribution state of the particle-like matter in the non-uniform-density sample.

3. An non-uniform-density sample analyzing method according to claim 1 or 2, wherein the fitting parameter indicates an average particle diameter and distribution shape of the particle-like matter, and the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the average particle diameter and the distribution shape of the particle-like matter in the non-uniform-density sample.

4. An-uniform-density sample analyzing method according to claim 1 or 2, wherein the fitting parameter indicates a nearest distance and correlation coefficient between the particle-like matter, and the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the nearest distance and correlation coefficient between the particle-like matters in the non-uniform-density sample.

5. An non-uniform-density sample analyzing method according to claim 1 or 2, wherein the fitting parameter indicates a content ratio and correlation distance of the particle-like matter, and the value of the fitting parameter when the simulated X-ray scattering curve agrees with the actually measured X-ray scattering curve or the value of the fitting parameter when the simulated particle beam scattering curve agrees with the actually measured particle beam scattering curve serves to indicate the content ratio and correlation distance of the particle-like matter in the non-uniform-density sample.

6. An non-uniform-density sample analyzing method according to any one of claims 1 to 5, wherein the actually measured X-ray scattering curve or the actually measured particle beam scattering curve is measured under any condition selected from the condition of θin=θout±offset angle Δω, condition of scanning θout with θin constant and condition for scanning θin with θout constant, and the simulated X-ray scattering curve or the simulated particle beam scattering curve is computed according to the scattering function under the same condition as that measuring condition.

7. An non-uniform-density sample analyzing method according to any one of claims 1 to 6, wherein a function which employs absorption/irradiating area correction taking into account at least one of refraction, scattering and reflection or particle-like matter correlation function or both of them is used as the scattering function.

8. An non-uniform-density sample analyzing method according to any one of claims 1 to 7, wherein the non-uniform-density sample is thin film or bulk body.

9. An non-uniform-density sample analyzing method according to claim 8, wherein the thin film is porous film and the particle-like matter is fine particle or pore forming the porous film.

10. An non-uniform-density sample analyzing device for analyzing distribution state of particle-like matter in a non-uniform-density sample, comprising:
a function storage means for storing a scattering function expressing a X-ray scattering curve according to a fitting parameter indicating distribution state of particle-like matter;
a simulating means for computing a simulated X-ray scattering curve under the same condition as a measuring condition of an actually measured X-ray scattering curve by using the scattering function from the function storage means; and
a fitting means for carrying out fitting between the simulated X-ray scattering curve and the actually measured X-ray scattering curve while changing the fitting parameter,
wherein the value of the fitting parameter when the simulated X-ray scattering curve agrees with the actually measured X-ray scattering curve serves to indicate the distribution state of the particle-like matter in the non-uniform-density sample.

11. An non-uniform-density sample analyzing device for analyzing distribution state of particle-like matter in a non-uniform-density sample, comprising:
a function storage means for storing a scattering function expressing a particle beam scattering curve according to a fitting parameter indicating distribution state of particle-like matter;
a simulating means for computing a simulated particle beam scattering curve under the same condition as a measuring condition of an actually measured particle beam scattering curve by using the scattering function from the function storage means; and
a fitting means for carrying out fitting between the simulated particle beam scattering curve and the actually measured particle beam scattering curve while changing the fitting parameter,
wherein the value of the fitting parameter when the simulated particle beam scattering curve agrees with the actually measured particle beam scattering curve serves to indicate the distribution state of the particle like matter in the non-uniform-density sample.

12. An non-uniform-density sample analyzing device according to claim 10 or 11, wherein when the actually measured X-ray scattering curve or the actually measured particle beam scattering curve is measured under any condition selected from the condition of θin=θout±offset angle Δω, condition of scanning θout with θin constant and condition of scanning θin with θout constant, the simulating means computes the simulated X-ray scattering curve or the simulated particle beam scattering curve with the scattering function under the same condition as that measuring condition.

13. An non-uniform-density sample analyzing device according to any one of claims 10 to 12, wherein the function storage means stores, as the scattering function, a function which employs absorption/irradiating area correction taking into account at least one of refraction, scattering and reflection or particle-like matter correlation function or both of them.

14. An non-uniform-density sample analyzing device according to any one of claims 10 to 13, wherein the non-uniform-density sample is thin film or bulk body.

15. An non-uniform-density sample analyzing device according to claim 14, wherein the thin film is porous film and the particle-like matter is fine particle or pore forming the porous film.

16. An non-uniform-density sample analyzing system for analyzing distribution state of particle-like matter in a non-uniform-density sample, comprising a X-ray measuring device for measuring an actually measured X-ray scattering curve in the non-uniform-density sample and the non-uniform-density sample analyzing device according to any one of claims 10, 12, 13, 14, 15, wherein the actually measured X-ray scattering curve by the X-ray measuring device and various kinds of parameters at the measurement necessary for computing the scattering function are made available by the non-uniform-density sample analysing device.

17. An non-uniform-density sample analyzing system for analyzing distribution state of particle-like matter in a non-uniform-density sample, comprising a particle beam measuring device for measuring an actually measured particle beam scattering curve in the non-uniform-density sample and the non-uniform-density sample analyzing device according to any one of claims 11, 12, 13, 14, 15, wherein the actually measured particle beam scattering curve by the particle beam measuring device and various kinds of parameters at measurement necessary for computing the scattering function are made available by the non-uniform-density sample analyzing device.

18. An non-uniform-density sample analyzing method for analyzing distribution state of particle-like matter in a non-uniform-density sample, characterized in that if the non-uniform-density sample is porous film, the distribution state of the particle-like matter in the porous film is analyzed using a measuring result of the X-ray scattering curve.
